(19) United States
(12) Patent Application Publication
(10) Pub. No.: US 2014/0236211 A1
(43) Pub. Date: Aug. 21, 2014

(54) MINIMALLY INVASIVE PULMONARY ARTERY BAND

(71) Applicants: KING ABDULLAH INTERNATIONAL MEDICAL RESEARCH CENTER, Riyadh (SA); KING SAUD BIN ABDULAZIZ UNIVERSITY FOR HEALTH SCIENCES, Riyadh (SA); NATIONAL GUARD HEALTH AFFAIRS, Riyadh (SA)

(72) Inventor: IMAD NAJA, RIYADH (SA)

(73) Assignees: KING ABDULLAH INTERNATIONAL MEDICAL RESEARCH CENTER, RIYADH (SA); NATIONAL GUARD HEALTH AFFAIRS, Riyadh (SA); KING SAUD BIN ABULAZIZ UNIVERSITY FOR HEALTH SCIENCES, Riyadh (SA)

(21) Appl. No.: 13/769,802

(22) Filed: Feb. 18, 2013

(51) Int. Cl. A61B 17/12

(52) U.S. Cl. CPC A61B 17/12013
USPC 606/195

(57) ABSTRACT

The minimally invasive pulmonary artery band includes an elongate tube having a reservoir of inflation fluid attached at one end and a clamp head attached to the other end. The clamp head is a hexagonal clamp frame having a gap to facilitate wrapping the clamp frame around the pulmonary artery. The clamp frame includes at least one balloon element attached thereto. The balloon element is selectively inflatable from the inflation fluid provided by the reservoir. The balloon element is configured for inflation into predefined shapes, and the pulmonary artery band allows for fine adjustments to compression and the shape of compression of the pulmonary artery to gradually reach a predefined state of occlusion.

### BACKGROUND OF THE INVENTION

1. Field of the Invention

The present invention relates to medical devices, and particularly to a minimally invasive pulmonary artery band that provides selective, controlled occlusion of the pulmonary artery in order to reduce hypertension and excessive pulmonary blood flow in postoperative patients.

2. Description of the Related Art

Ideally, it is a common wish that all infants will be born healthy and without defect. Unfortunately, circumstances are not ideal. While many infants are born without serious health issues, some are born with congenital defects, such as abnormalities relating to the heart. One such condition promotes excessive pulmonary blood flow, which places excessive pressure on the pulmonary artery. This can lead to malformed lungs, lesions in the lungs causing respiratory problems, feeding difficulties, and stunted growth.

Several palliative solutions have been proposed to combat these issues. One solution involves suturing the pulmonary artery to reduce blood flow. This is a risky procedure, and there is no guarantee the sutures will stay long enough to normalize blood flow. Premature tearing of the sutures can also cause further complications.

Another solution involves wrapping a tape around the arterial branch. This requires superb skill on the part of the surgeon in order to avoid excessive distortion of the artery. Moreover, fine adjustments to the constriction cannot be easily accomplished.

A still further solution involves vascular occlusion clamps. These types of devices are usually a clamp-shaped element having an inflatable balloon attached therein. The clamp element surrounds the target artery and the balloon is inflated to obtain the desired occlusion. For the most part, this solution works well. However, such occlusion clamps tend to be very limited in the geometry of the inflated balloon. The balloon usually forms an annular or ovoid ring around the artery. It has been found that different constriction geometries can have significant impact on the pressure and blood flow of the artery. Thus, while conventional vascular occlusion clamps may be satisfactory for most applications, there appears to be problems in obtaining the constrictive geometry required to accommodate the specific conditions the surgeon desires to address.

In light of the above, it would be a benefit in the medical arts to provide a vascular occlusion clamp that provides fine adjustments to inflation geometry and pressure for optimizing recovery of postoperative patients. Thus, a minimally invasive pulmonary artery band solving the aforementioned problems is desired.

### SUMMARY OF THE INVENTION

The minimally invasive pulmonary artery band includes an elongate tube with a reservoir attached at one end and a clamp head attached to the other end. The clamp head is a hexagon-shaped clamp frame having a gap to facilitate wrapping the clamp frame around the pulmonary artery. The clamp frame includes at least one balloon element attached thereto. The balloon element is selectively inflatable with inflation fluid provided by the reservoir. The balloon element is configured for inflation into predefined shapes, and the pulmonary artery band allows for fine adjustments to gradually reach a predefined state of occlusion.

These and other features of the present invention will become readily apparent upon further review of the following specification and drawings.

### BRIEF DESCRIPTION OF THE DRAWINGS

FIG. 1 is an environmental, perspective view of a minimally invasive pulmonary artery band according to the present invention.

FIG. 2 is a perspective view of the minimally invasive pulmonary artery band of FIG. 1.

FIG. 3A is a schematic diagram of the minimally invasive pulmonary artery band of FIGS. 1 and 2 shown in an operative position around the pulmonary artery, shown before inflation of the balloon.

FIGS. 3B, 3C, and 3D are schematic diagrams of the various possible inflation geometries for the minimally invasive pulmonary artery band of FIG. 3A after inflation of the balloon.

Similar reference characters denote corresponding features consistently throughout the attached drawings.

### DETAILED DESCRIPTION OF THE PREFERRED EMBODIMENTS

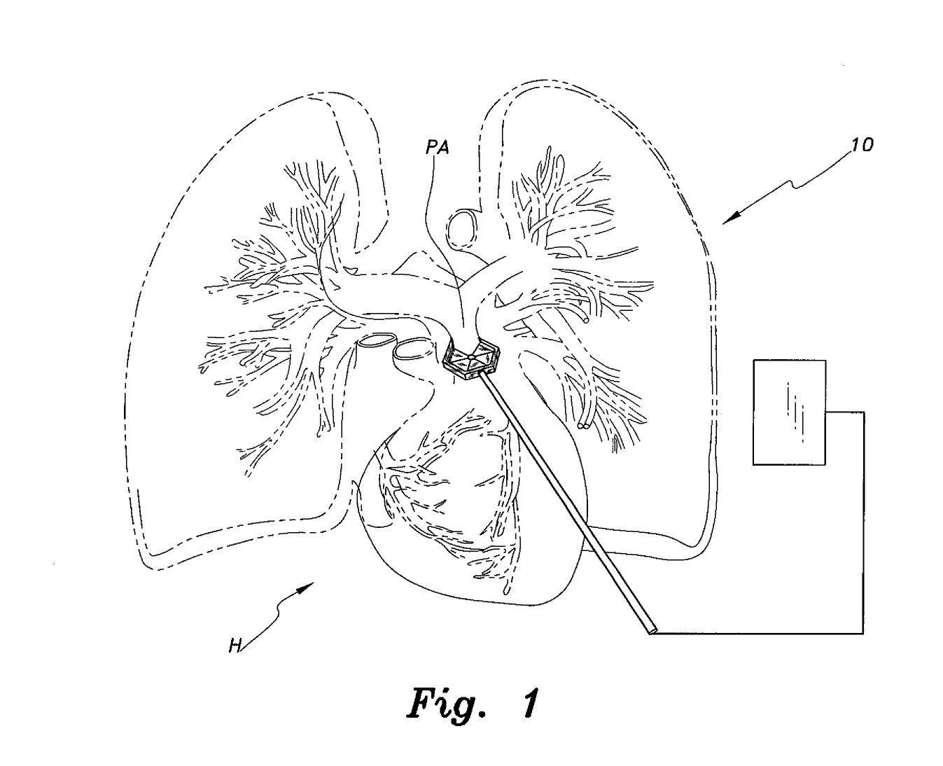

The minimally invasive pulmonary artery band, generally referred to in the drawings by the reference number 10, provides fine adjustable control of vascular occlusion to optimize postoperative recovery by minimizing some of the complications that may arise from such surgery. The artery band 10 is configured as a surgically implanted device with external access to the inflation mechanism. It is noted that the pulmonary artery band 10 is preferably used on the main pulmonary artery PA of the heart H in most cases. The perspective in FIG. 1 is to best show the pulmonary artery band 10 in use. It should also be recognized that the pulmonary artery band 10 can be placed anywhere where minimally invasive occlusion is desired.

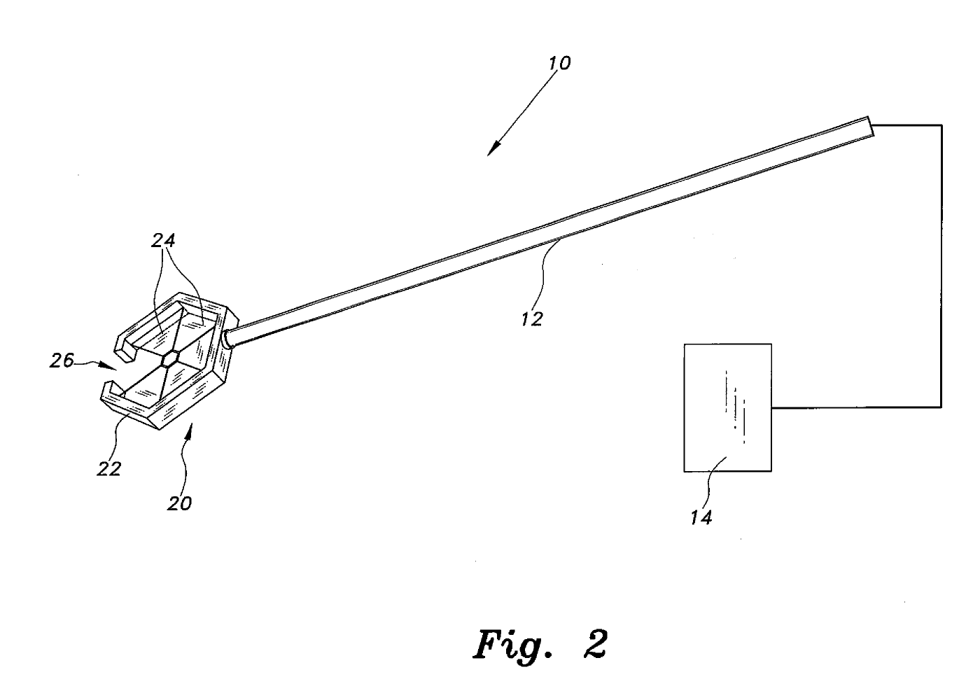

As best seen in FIGS. 1 and 2, the minimally invasive pulmonary artery band 10 includes an elongate tube 12 having one end selectively attached to an inflation system 14 and the opposite attached to a clamp head 20. Both the tube 12 and the clamp head 20 are constructed from biocompatible materials. The tube 12 is preferably about 200 mm long and has an internal diameter of about 0.200 mm.

The inflation system 14 includes a reservoir of saline solution or the like, which serves as the medium for inflation and/or a mechanism for introducing the solution to the clamp head 20. The mechanism can include injectors, such as a syringe, pumps and the like.

The clamp head 20 includes a clamp frame 22 and at least one balloon element 24 attached to the clamp frame 22. The balloon element 24 is also constructed from biocompatible materials. In this embodiment, the clamp frame 22 is constructed as a hexagon, and the balloon element 24 extends radially inward from five of the six sides of the hexagon. A gap 26 is formed in one side of the hexagon, the gap 26 permitting passage of the clamp frame 22 around the pulmonary artery PA. The balloon element 24 is substantially encapsulated inside the clamp frame 22 and divided into segments. Each segment of the balloon 24 forms a substantially triangular wedge-shape when fully inflated. Inflation of the balloon element 24 can be facilitated by dispersal of the inflation fluid to each segment or to selected segments of the balloon elements 24 through the clamp frame 22, by inter-communication of the segments to permit inflation fluid flow from one segment to another, or by a combination thereof. The clamp frame 22 is preferably about 4.000 mm thick and about 15.000 mm wide, measured from opposing sides of the hexagon.

In use, the clamp head 20 is placed around the pulmonary artery PA by slipping the pulmonary artery PA through the gap 26 in the clamp frame 22, and the balloon element 24 is selectively inflated to an initial degree via the inflation system 14. Unlike most conventional vascular occlusion devices, the minimally invasive pulmonary artery band 10 allows for fine control of the constriction. The postoperative period is a delicate time where blood pressure, right ventricle pressure, and pulmonary artery pressure, as well as oxygen saturation, may cause serious threats to neonatal patients. Any complications from the above may require drug therapy or additional operation to make the necessary adjustments. In order to reduce such complications, the doctor or user can adjust the compression or decompression of the artery PA to the target occlusion parameters in a safer, gradual manner. For example, if a 50% pulmonary artery diameter reduction is desired, then the user can increase the inflation of the balloon element 24 to provide a 5-10% diameter reduction every 2-3 days, which provides time for recovery and stabilization. On the other hand, a sudden 50% reduction can have detrimental effects on the patient's respiratory system.

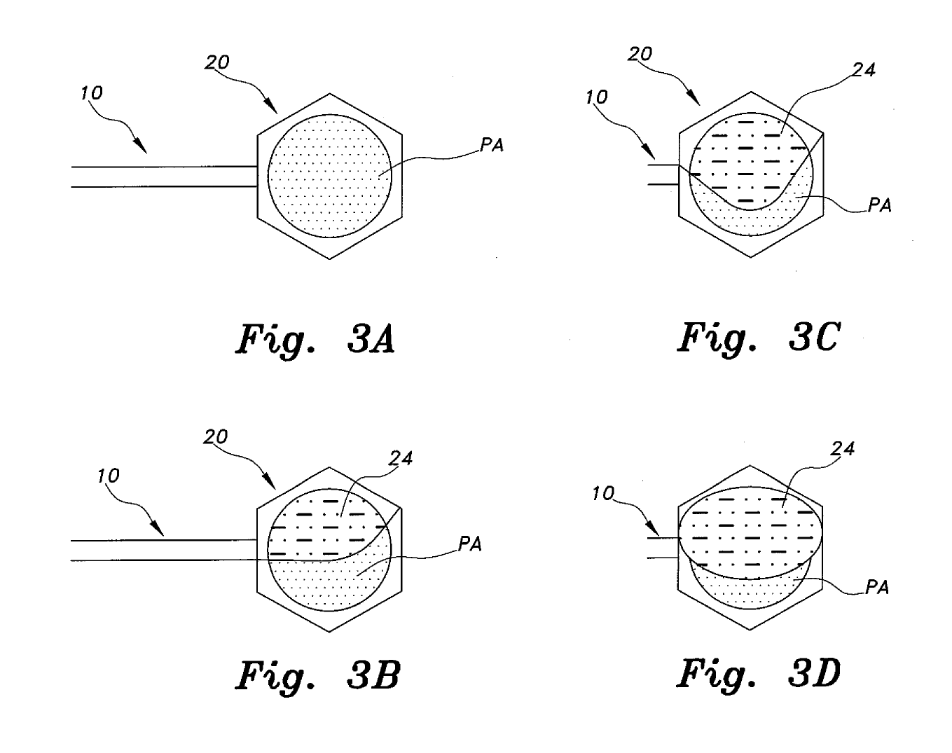

In addition to the above, the inflation characteristic and geometry (shape) of the balloon element 24 can also be controlled to facilitate specific shaping of the artery in order to control the blood flow rate and pressure. FIGS. 3A-3D show some of the possible configurations for the minimally invasive pulmonary artery band 10.

FIG. 3A schematically shows the clamp head 20 surrounding the pulmonary artery PA before inflation of the balloon. FIG. 3B shows a substantially diagonal inflation of the balloon elements 24, resulting in a substantially rounded wedge. FIG. 3C shows inflation substantially from the top-down, resulting in a similar rounded wedge-shape. FIG. 3D shows an ovoid inflation from the top section of the clamp head 20. It is to be understood that any of these inflation characteristics, shapes and variations thereof can originate from any side or combination of sides of the clamp frame 22, i.e. top-down, bottom-up, side-to-side, etc.

It is to be understood that the present invention is not limited to the embodiments described above, but encompasses any and all embodiments within the scope of the following claims.

I claim:

1. A minimally invasive pulmonary artery band, comprising:
an inflation system having a reservoir of inflation fluid, the inflation system being accessible by a user externally of a patient;
a clamp head attached to the other end of the elongate tube, the clamp head being configured to surround the patient's pulmonary artery, the clamp head having a hexagonal clamp frame having a gap formed in one side thereof, the gap permitting passage of the pulmonary artery in order to have the clamp frame substantially surround the pulmonary artery;
an elongate tube extending between the inflation system and the clamp head; and
at least one balloon element attached to the clamp frame, the tube being connected to the at least one balloon element, the at least one balloon element being selectively inflatable by the inflation system into a specific shape in order to occlude the pulmonary artery by compressing the pulmonary artery into a desired shape for predefined blood flow and pressure;
wherein fine inflation adjustments can be made to the at least one balloon element by the inflation system to gradually obtain a predefined target occlusion of the pulmonary artery.

2. The minimally invasive pulmonary artery band according to claim 1, wherein said elongate tube and said clamp head are constructed from biocompatible materials.

3. The minimally invasive pulmonary artery band according to claim 1, wherein said inflation fluid comprises a saline solution.

4. The minimally invasive pulmonary artery band according to claim 1, wherein said elongate tube comprises a biocompatible cylindrical tube having a length of about 200 mm and an internal diameter of about 0.200 mm.

5. The minimally invasive pulmonary artery band according to claim 1, wherein said hexagonal clamp frame measures about 15 mm between opposing sides.

6. The minimally invasive pulmonary artery band according to claim 1, wherein said at least one balloon element forms a substantially rounded wedge-shape extending from a section of said clamp frame when inflated.

7. The minimally invasive pulmonary artery band according to claim 1, wherein said at least one balloon element forms a substantially ovoid shape extending from a section of said clamp frame when inflated.

8. A method of forming a vascular occlusion in a pulmonary artery, the method comprising the steps of:
providing a minimally invasive pulmonary artery band, the minimally invasive pulmonary band having;
an inflation system having a reservoir of inflation fluid, the inflation system being accessible by a user externally of a patient;
a clamp head attached to the other end of the elongate tube, the clamp head being configured to surround the patient's pulmonary artery, the clamp head having a hexagonal clamp frame having a gap formed in one side thereof, the gap permitting passage of the pulmonary artery in order to have the clamp frame substantially surround the pulmonary artery;
an elongate tube extending between the inflation system and the clamp head; and
at least one balloon element attached to the clamp frame, the tube being connected to the at least one balloon element, the at least one balloon element being selectively inflatable by the inflation system into a specific shape in order to occlude the pulmonary artery by compressing the pulmonary artery into a desired shape for predefined blood flow and pressure;
implanting the minimally invasive pulmonary artery band so that the clamp head surrounds the pulmonary artery;
inflating the at least one balloon element with the inflation system to an initial amount to provide a fraction of a predetermined occlusion amount; and
gradually increasing inflation of the at least one balloon element at set intervals of time till the predetermined occlusion parameters have been met.
